(19) United States
(12) Patent Application Publication
(10) Pub. No.: US 2018/0044348 A1
(43) Pub. Date: Feb. 15, 2018

(54) CRYSTALLINE SALTS OF MORPHINE SULFATE

(71) Applicant: Grünenthal Gmbh, Aachen (DE)

(72) Inventors: Klaus WENING, Koln (DE); Lutz BARNSCHEID, Monchengladbach (DE); Anja GEISSLER, Stolberg (DE); Jana DENKER, Bornheim (DE); Dagmar LISCHKE, Inden-Altdorf (DE)

(73) Assignee: Grünenthal Gmbh, Aachen (DE)

(21) Appl. No.: 15/791,563

(22) Filed: Oct. 24, 2017

Related U.S. Application Data
(63) Continuation of application No. PCT/EP2016/025041, filed on Apr. 22, 2016.

(30) Foreign Application Priority Data
Apr. 24, 2015 (EP) 15001226.8

(51) Int. Cl. C07D 489/04

(52) U.S. Cl. CPC C07D 489/04; C07B 2200/13

(57) ABSTRACT

Crystalline salts of 7,8-Didehydro-4,5 alpha-epoxy-17-methylmorphinan-3,6 alpha-diol sulfate(2:1), i.e. of morphine sulphate, are disclosed and four polymorphic forms of these salts are reported. The invention further relates to a pharmaceutical composition comprising an amount of such a salt and such a salt as a medicament and for the treatment and/or prevention of pain.

This application is a continuation of PCT/EP2016/025041, filed Apr. 22, 2016, which claims foreign priority benefit under 35 U.S.C. §119 of the European Patent Application No. 15001226.8, filed Apr. 24, 2015, the disclosures of which are incorporated herein by reference.

The present invention relates to a crystalline salt of morphine, i.e. of 7,8-Didehydro-4,5 alpha-epoxy-17-methylmorphinan-3,6 alpha-diol sulfate(2:1). The invention further relates to a pharmaceutical composition comprising an amount of such a salt and such a salt as a medicament and for the treatment and/or prevention of pain.

Morphine sulfate, generally administered in the form of its pentahydrate salt, is a potent analgesic, for the relief of moderate to severe pain. In fact, it may act as an analgesic. This salt can be administered as an injectable solution, suppository, tablet or extended release tablet.

Crystalline salts and thus polymorphs of this kind of analgesic are to best of knowledge not known in the prior art.

As a rule, the crystalline state of an active pharmaceutical substance has the advantage of an increased stability and processability.

It would therefore be desirable to have access to crystalline forms of the sulfate pentahydrate salt of morphine sulfate. The present invention has the object of addressing these needs and of providing such morphine salts.

According to the present invention this object is achieved by a crystalline, salt of 7,8-Didehydro-4,5 alpha-epoxy-17-methylmorphinan-3,6 alpha-diol sulfate(2:1) according to formula (I):

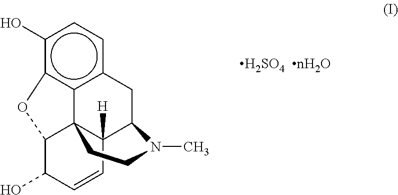

Formula (I) thus describes 7,8-Didehydro-4,5 alpha-epoxy-17-methylmorphinan-3,6 alpha-diol sulfate(2:1) (salt), or, in the sense of the present invention, morphine sulphate.

According to the above formula (I), depending on the respective embodiments, it may be provided that the crystalline salt is present in a hydrate form or in an anhydrous form. Correspondingly, it is provided that 0≦n≦5, wherein in the case that n=0, the crystalline form is an anhydrate, whereas in case n>0, the crystalline salt is present in a hydrate form. Regarding the hydrate form, it may be provided that n=5, and the salt may thus be present as a pentahydrate. However, it may under circumstances as well be the case that n lies above 0 but below 5.

It may further be provided that either one or two protons of the morphine structure may be dissociated and the respective oxygen is then coupled to a sulfate structure.

The compounds according to the invention are present in a crystalline state which is conveniently defined as the presence of one or preferably more reflexes in an X-ray powder diffractogram. In this respect the term “X-ray crystalline” is also used.

It has also been discovered that the crystalline salts of the present invention may be obtained in several polymorphic forms. Polymorphism, the occurrence of different crystal forms, is a property of some molecules and molecular complexes. A single compound may give rise to a variety of polymorphs having distinct crystal structures and physical properties like melting point, x-ray diffraction pattern, infrared absorption fingerprint, and solid state NMR spectrum. One polymorph may give rise to thermal behavior different from that of another polymorph.

The difference in the physical properties of different polymorphs results from the orientation and intermolecular interactions of adjacent molecules or complexes in the bulk solid. Accordingly, polymorphs are distinct solids sharing the same molecular formula yet having distinct advantageous physical properties compared to other polymorphs of the same composition or complex.

The discovery of new polymorphic forms and solvates of a pharmaceutically useful composition provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic. Therefore, there is a need for additional polymorphs of morphine sulfate.

The polymorphic forms according to the present invention may have significant advantages over the prior art and especially over potential amorphous forms of morphine sulfate.

In fact, the polymorphic forms provided, especially when used as a medicament, may show advantages with regard to efficiency when administered to a patient e.g. due to a good bioavailability as well as with regard to stability when stored. For example, the polymorphic forms of the present invention may have advantages with regard to medicaments showing delayed release.

According to a first embodiment, the X-ray powder diffractogram (XRPD) of the salt comprises one or more of the following reflexes (±0,2 in 2θ (2 theta)): 13.6, 15.2, 17.7. Preferably, the following reflexes (±0,2 in 2θ) are included: 13.6, 14.2, 15.2, 17.7, 24.2. More preferably, the following reflexes (±0,2 in 2θ) are included: 8.4, 12.1, 12.5, 13.6, 14.2, 15.2, 16.3, 16.7, 16.9, 17.3, 17.7, 20.0, 20.4, 21.0, 24.2, 24.5, 25.2, 25.5, 25.9, 26.4, 28.4, 29.1, 30.0, 30.7, 31.3, 32.0, 32.9, 33.7. In the context of the present invention this polymorph will be referred to as the “first polymorph”.

It is most preferred that its X-ray powder diffractogram comprises one or more of the following reflexes as stated in the following table 1, or it may consist of the following reflexes as stated in the following table 1 (±0,2 in 2θ and recorded using CuK_{α} radiation; the relative intensity I(rel) is 100 at most).

TABLE 1
X-ray powder diffractogram of the first polymorph

| 2θ | I(rel) |
|---|---|
| 8.4 | 24.5 |
| 12.1 | 8.8 |
| 12.5 | 17.4 |
| 13.6 | 56.8 |
| 14.2 | 45.9 |
| 15.2 | 55.2 |
| 16.3 | 17.0 |
| 16.7 | 20.5 |
| 16.9 | 24.4 |
| 17.3 | 9.3 |
| 17.7 | 46.1 |
| 20.0 | 22.8 |
| 20.4 | 19.8 |
| 21.0 | 22.7 |
| 24.2 | 30.1 |
| 24.5 | 21.3 |
| 25.2 | 9.2 |
| 25.5 | 16.9 |
| 25.9 | 19.3 |
| 26.4 | 15.0 |
| 28.4 | 7.6 |
| 29.1 | 10.1 |
| 30.0 | 5.2 |
| 30.7 | 8.9 |
| 31.3 | 4.7 |
| 32.0 | 6.0 |
| 32.9 | 7.8 |
| 33.7 | 7.0 |

The polymorphic form of morphine sulfate, i.e. of the salt of 7,8-Didehydro-4,5 alpha-epoxy-17-methylmorphinan-3,6 alpha-diol sulfate(2:1) of this first embodiment is present in a hydrate form, i.e. it substantially has five or less molecules of water (H₂O) in its crystal structure for each molecule of morphine sulphate. It may be formed as follows:

Firstly, a formulation is provided, which comprises commercially available morphine sulfate pentahydrate and different further components. The formulation may be formed by weighing and blending the respective compounds. A further step of sieving may further be realized, for example before blending, in order to separate large impurities or agglomerated parts, for example. The formulation may then be extruded by use of an extruder, especially by use of a twin screw extruder. The product being formed in the extruder comprises the first polymorph. Therefore, the first polymorph is present in a formulation.

With regard to the extruder, the latter may comprises different heating zones which are arranged one after the other in a flow direction of the extruded material. For example, a temperature profile may be used which provides a, preferably slight, decrease with regard to temperature in the forward direction of the extruded material. The temperature in the extruder may be raised to essentially 90° C. to 120° C. or above, referring to the heating element of the heating zone with the highest temperature at which the temperature decrease starts. The first temperature at the entrance of the extruder may further have a lower temperature then the second temperature zone, which may then be heated as described above and after which a temperature decrease may follow. Regarding the temperature used, the first heating zone especially at the feeding portion of the extruder may lie in a range of 20° C. to 30° C., wherein the further heating zones, excepting the nozzle lying at the end of the extruder, which may have a temperature in the range of 120° C. to 150° C., may lie in a range of 40° C. to 120° C., wherein, as described above, a temperature decrease may be realized.

Regarding the second embodiment, it is provided that the X-ray powder diffractogram (XRPD) of the salt comprises one or more of the following reflexes (±0,2 in 2θ (2 theta)): 13.1, 14.2, 16.3. Preferably, the following reflexes (±0,2 in 2θ) are included: 12.4, 13.1, 14.2, 16.3, 17.3, 20.1. More preferably, the following reflexes (±0,2 in 2θ) are included: 9.1, 12.4, 13.1, 14.2, 16.3, 17.3, 20.1, 24.2, 24.7. In the context of the present invention this polymorph will be referred to as the “second polymorph”.

It is most preferred that its X-ray powder diffractogram comprises one or more of the following reflexes as stated in the following table 2, or it may consist of the following reflexes as stated in the following table 2 (±0,2 in 2θ and recorded using CuK_{α} radiation; the relative intensity I(rel) is 100 at most):

TABLE 2
X-ray powder diffractogram of the second polymorph according to the
second embodiment

| 2θ | I (rel) |
|---|---|
| 9.1 | 10.9 |
| 12.4 | 16.4 |
| 13.1 | 52.0 |
| 14.2 | 74.1 |
| 16.3 | 32.0 |
| 17.3 | 21.0 |
| 20.1 | 12.1 |
| 24.2 | 8.5 |
| 24.7 | 4.9 |

The polymorphic form of morphine sulfate, i.e. of the salt 7,8-Didehydro-4,5 alpha-epoxy-17-methylmorphinan-3,6 alpha-diol sulfate(2:1) of this second embodiment may be formed as follows:

Firstly, a formulation is provided, which comprises commercially available morphine sulfate pentahydrate and different further components. The formulation may be formed by weighing and blending the respective compounds. A further step of sieving may further be realized, for example before blending, in order to separate large impurities or agglomerated parts, for example. The formulation may then be extruded by use of an extruder, especially by use of a twin screw extruder. The product being formed in the extruder comprises the third polymorph. Therefore, the first polymorph is present in a formulation.

With regard to the extruder, the latter may comprise different heating zones which are arranged one after the other in a flow direction of the extruded material. For example, a temperature profile may be used which provides a, preferably slight, decrease with regard to temperature in the forward direction of the extruded material. The temperature in the extruder may be raised to above 100° C. referring to the heating elements. Regarding the temperature used, the first heating zone especially at the feeding portion of the extruder may lie in a range of 20° C. to 35° C., wherein the further heating zones may lie in a range of 80° C. to 160° C., wherein, as described above, a temperature increase may be realized.

Regarding the third embodiment, it is provided that the X-ray powder diffractogram (XRPD) of the salt comprises one or more of the following reflexes (±0,2 in 2θ (2 theta)): 14.2, 16.4, 20.1. Preferably, the following reflexes (±0,2 in 2θ) are included: 14.2, 16.4, 17.3, 20.1, 20.6. More preferably, the following reflexes (±0,2 in 2θ) are included: 9.1, 12.4, 14.2, 16.4, 17.3, 17.8, 20.1, 20.6. In the context of the present invention this polymorph again is the “second polymorph”.

It is most preferred that its X-ray powder diffractogram comprises one or more of the following reflexes as stated in the following table 3, or it may consist of the following reflexes as stated in the following table 3 (±0,2 in 2θ and recorded using CuK_{α} radiation; the relative intensity I(rel) is 100 at most):

TABLE 3
X-ray powder diffractogram of the second polymorph and according
to the third embodiment

| 2θ | I (rel) |
|---|---|
| 9.1 | 1.0 |
| 12.4 | 1.5 |
| 14.2 | 7.3 |
| 16.4 | 3.9 |
| 17.3 | 2.0 |
| 17.8 | 1.9 |
| 20.1 | 2.8 |
| 20.6 | 2.3 |

With regard to a comparison of the second embodiment, and the third embodiment and thus a comparison of the second polymorph in two formulations, it may be provided that special peaks are present or not and the relative intensities may vary. This may be due to an interaction of components being present in the formulation leading to potential differences of the respective spectra.

The polymorphic form of morphine sulfate, i.e. of the salt of 7,8-Didehydro-4,5 alpha-epoxy-17-methylmorphinan-3,6 alpha-diol sulfate(2:1) of this third embodiment and thus the second polymorph may be formed as follows:

Firstly, a formulation is provided, which comprises commercially available morphine sulfate pentahydrate and different further components. The formulation may be formed by weighing and blending the respective compounds. A further step of sieving may further be realized, for example before blending, in order to separate large impurities or agglomerated parts, for example. The formulation may then be extruded by use of an extruder, especially by use of a twin screw extruder. The product being formed in the extruder comprises the third polymorph. Therefore, the first polymorph is present in a formulation.

With regard to the extruder, the latter may comprise different heating zones which are arranged one after the other in a flow direction of the extruded material. For example, a temperature profile may be used which provides a, preferably slight, increase with regard to temperature in the forward direction of the extruded material. The temperature in the extruder may be raised to above 100° C. referring to the heating elements. Regarding the temperature used, the first heating zone especially at the feeding portion of the extruder may lie in a range of 20° C. to 30° C., wherein the further heating zones may lie in a range of 80° C. to 150° C., wherein, as described above, a temperature increase may be realized.

Regarding the fourth embodiment, it is provided that the X-ray powder diffractogram (XRPD) of the salt comprises one or more of the following reflexes (±0,2 in 2θ (2 theta)): 13.0, 14.9, 31.7. Preferably, the following reflexes (±0,2 in 2θ) are included: 13.0, 14.3, 14.9, 18.3, 31.7. More preferably, the following reflexes (±0,2 in 2θ) are included: 11.9, 13.0, 14.3, 14.9, 16.1, 16.4, 18.3, 31.7. In the context of the present invention this polymorph will be referred to as the “third polymorph”.

It is most preferred that its X-ray powder diffractogram comprises one or more of the following reflexes as stated in the following table 4, or it may consist of the following reflexes as stated in the following table 4 (±0,2 in 2θ and recorded using CuK_{α} radiation; the relative intensity I(rel) is 100 at most):

TABLE 4
X-ray powder diffractogram of the third polymorph

| 2θ | I (rel) |
|---|---|
| 11.9 | 2.4 |
| 13.0 | 5.0 |
| 14.3 | 3.9 |
| 14.9 | 5.4 |
| 16.1 | 1.4 |
| 16.4 | 2.1 |
| 18.3 | 3.2 |
| 31.7 | 5.1 |

The polymorphic form of morphine sulfate, i.e. of the salt of 7,8-Didehydro-4,5 alpha-epoxy-17-methylmorphinan-3,6 alpha-diol sulfate(2:1) of this fourth embodiment is present in an anhydrate form or in a hydrate form having five or less molecules of water (H₂O) in its crystal structure for each molecule of morphine sulphate. It may be formed as follows:

Firstly, a formulation is provided, which comprises commercially available morphine sulfate pentahydrate and different further components. The formulation may be formed by weighing and blending the respective compounds. A further step of sieving may further be realized, for example before blending, in order to separate large impurities or agglomerated parts, for example. The formulation may then be extruded by use of an extruder, especially by use of a twin screw extruder. The product being formed in the extruder comprises the third polymorph. Therefore, the first polymorph is present in a formulation.

With regard to the extruder, the latter may comprise different heating zones which are arranged one after the other in a flow direction of the extruded material. For example, a temperature profile may be used which provides a, preferably slight, increase with regard to temperature in the forward direction of the extruded material. The temperature in the extruder may be raised to above 100° C. referring to the heating elements. Regarding the temperature used, the first heating zone especially at the feeding portion of the extruder may lie in a range of 20° C. to 30° C., wherein the further heating zones may lie in a range of 80° C. to 150° C., wherein, as described above, a temperature increase may be realized.

According to the fifth embodiment, it is provided that the X-ray powder diffractogram (XRPD) of the salt comprises one or more of the following reflexes (±0,2 in 2θ (2 theta)): 12.3, 14.0, 16.2. Preferably, the following reflexes (±0,2 in 2θ) are included: 12.3, 14.0, 16.2, 16.8, 20.6, 25.1. More preferably, the following reflexes (±0,2 in 2θ) are included: 6.1, 10.5, 10.9, 11.7, 12.3, 13.1, 14.0, 15.7, 16.2, 16.8, 17.6, 20.6, 21.2, 22.0, 25.1, 25.8, 28.5, 29.1, 29.8. In the context of the present invention this polymorph will be referred to as the “fourth polymorph”.

It is most preferred that its X-ray powder diffractogram comprises one or more of the following reflexes as stated in the following table 5, or it may consist of the following reflexes as stated in the following table 5 (±0,2 in 2θ and recorded using CuK_{α} radiation; the relative intensity I(rel) is 100 at most):

TABLE 5
X-ray powder diffractogram of the fourth polymorph according to the fifth
embodiment

| 2θ | I (rel) |
|---|---|
| 6.1 | 13.2 |
| 10.5 | 10.9 |
| 10.9 | 14.4 |
| 11.7 | 2.0 |
| 12.3 | 68.6 |
| 13.1 | 8.8 |
| 14.0 | 94.7 |
| 15.7 | 5.6 |
| 16.2 | 48.6 |
| 16.8 | 17.1 |
| 17.6 | 15.6 |
| 20.6 | 37.1 |
| 21.2 | 4.2 |
| 22.0 | 9.8 |
| 25.1 | 18.3 |
| 25.8 | 13.4 |
| 28.5 | 7.2 |
| 29.1 | 2.8 |
| 29.8 | 4.5 |

The polymorphic form of morphine sulfate, i.e. of the salt of 7,8-Didehydro-4,5 alpha-epoxy-17-methylmorphinan-3,6 alpha-diol sulfate(2:1) of this fifth embodiment may be formed as follows:

Firstly, the extrudate according to the above-defined first embodiment is generated. Such extrudate was processed in a DVS experiment (Dynamic-Vapour-Sorption). The extrudate was processed with a distinct humidity program. The probe was processed at a comparably higher relative humidity, e.g. 80% rH to 95% rH, for example at 90% rH for a time range of 10 to 15 hours, e.g. of 12 hours. Afterwards, the humidity was decreased and the probe was processed at a comparably lower relative humidity, e.g. 5% rH to 15% rH, for example at 10% rH for a time range of 10 to 15 hours, e.g. of 12 hours. The formed substance was morphine sulfate according to the fifth embodiment. As such, the form provided was present in a formulation. The device used was a DVS-1000, commercially available by the firm Surface Measurement Systems.

According to the sixth embodiment, it is provided that the X-ray powder diffractogram (XRPD) of the salt comprises one or more of the following reflexes (±0,2 in 2θ (2 theta)): 14.0, 14.2, 20.7. Preferably, the following reflexes (±0,2 in 2θ) are included: 12.3, 14.0, 14.2, 16.2, 20.7, 25.8. More preferably, the following reflexes (±0,2 in 2θ) are included: 11.0, 12.3, 13.1, 14.0, 14.2, 16.2, 16.8, 17.7, 19.0, 20.7, 22.7, 24.9, 2-5.2, 25.8, 28.5. More preferably, the following reflexes (±0,2 in 2θ) are included: 6.1, 10.5, 11.0, 12.3, 13.1, 14.0, 14.2, 15.8, 16.2, 16.8, 17.7, 18.7, 19.0, 20.7, 21.3, 22.0, 22.7, 23.1, 24.5, 24.9, 25.2, 25.5, 25.8, 26.4, 27.4, 27.7, 28.0, 28.5, 29.2, 29.9, 30.5, 30.8, 31.0, 31.9, 32.8, 33.4, 35.4, 35.9, 37.6, 39.8, 41.5. In the context of the present invention this polymorph will again be referred to as the “fourth” polymorph, which is present in an isolated form.

It is most preferred that its X-ray powder diffractogram comprises one or more of the following reflexes as stated in the following table 6, or it may consist of the following reflexes as stated in the following table 6 (±0,2 in 2θ and recorded using CuK_{α} radiation; the relative intensity I(rel) is 100 at most):

TABLE 6
X-ray powder diffractogram of the fourth polymorph according to the sixth
embodiment

| 2θ | I (rel) |
|---|---|
| 6.1 | 6.9 |
| 10.5 | 8.9 |
| 11.0 | 10.1 |
| 12.3 | 43.1 |
| 13.1 | 17.5 |
| 14.0 | 67.3 |
| 14.2 | 100 |
| 15.8 | 7.5 |
| 16.2 | 35.4 |
| 16.8 | 26.8 |
| 17.7 | 21.7 |
| 18.7 | 5.3 |
| 19.0 | 15.4 |
| 20.7 | 48.5 |
| 21.3 | 3.2 |
| 22.0 | 3.6 |
| 22.7 | 28.5 |
| 23.1 | 8.5 |
| 24.5 | 1.9 |
| 24.9 | 14.6 |
| 25.2 | 23.4 |
| 25.5 | 8.7 |
| 25.8 | 38.4 |
| 26.4 | 5.0 |
| 27.4 | 2.2 |
| 27.7 | 8.1 |
| 28.0 | 7.8 |
| 28.5 | 14.6 |
| 29.2 | 1.4 |
| 29.9 | 3.3 |
| 30.5 | 1.5 |
| 30.8 | 4.6 |
| 31.0 | 5.7 |
| 31.9 | 2.3 |
| 32.8 | 3.5 |
| 33.4 | 6.9 |
| 35.4 | 2.9 |
| 35.9 | 4.4 |
| 37.6 | 3.0 |
| 39.8 | 2.7 |
| 41.5 | 8.6 |

The polymorphic form of morphine sulfate, i.e. of the salt of 7,8-Didehydro-4,5 alpha-epoxy-17-methylmorphinan-3,6 alpha-diol sulfate(2:1) of this sixth embodiment may be formed as follows:

Morphine sulfate pentahydrate was processed in a DVS experiment (Dynamic-Vapour-Sorption. The morphine sulfate pentahydrate used may be commercially available morphine sulfate pentahydrate. It was processed with a distinct humidity program. The probe was processed at a comparably higher relative humidity, e.g. 80% rH to 95% rH, for example at 90% rH for a time range of 10 to 15 hours, e.g. of 12 hours. Afterwards, the humidity was decreased and the probe was processed at a comparably lower relative humidity, e.g. 5% rH to 15% rH, for example at 10% rH for a time range of 10 to 15 hours, e.g. of 12 hours. The formed substance was morphine sulfate according to the fifth embodiment. As such, the form provided was present in an isolated form. The device used was a DVS-1000, commercially available by the firm Surface Measurement Systems.

A further aspect of the present invention is a pharmaceutical composition comprising an especially effective amount of a crystalline salt of 7,8-Didehydro-4,5 alpha-epoxy-17-methylmorphinan-3,6 alpha-diol sulfate(2:1) of formula (I) according to the invention.

The quantity of the crystalline morphine sulfate amounting to an amount, particularly to an effective amount, of this substance depends substantially on the type of formulation and on the desired dosage during the period of administration. The quantity of the respective compounds to be administered to the patient may vary and is, for example, dependent on the weight or age of the patient and also on the manner of administration, on the indication and on the degree of severity of the illness.

Oral formulations may be solid formulations, for example tablets, capsules, pills and pastilles, but oral formulations may also be liquid formulations, for example solutions, suspensions, syrups or elixirs. Liquid and solid formulations also encompass the incorporation of the crystalline, morphine sulfate into solid or liquid foodstuffs. Furthermore, liquids also encompass solutions for parenteral applications, such as, for example, solutions for infusion or injection.

The crystalline morphine sulfate can be used directly as powders (micronized particles), granulates, suspensions or solutions, or they may be mixed with other pharmaceutically acceptable ingredients and components and then pulverized, in order then to fill the powders into capsules consisting of hard or soft gelatin, to press tablets, pills or pastilles, or in order to suspend or dissolve the powders in a carrier for the purpose of preparing suspensions, syrups or elixirs. Tablets, pills or pastilles can be provided with a coating after pressing.

Pharmaceutically acceptable ingredients and components for the various types of formulation are known as such. It may, for example, be a question of binding agents such as synthetic or natural polymers, medicinal carriers, lubricating agents, surfactants, sweetening agents and flavoring agents, coating agents, preserving agents, dyestuffs, thickening agents, ancillary agents, antimicrobial agents and carriers for the various types of formulation, as they are generally known in the art.

Examples of suitable binding agents include gum arabic, gum tragacanth, acacia gum and biodegradable polymers such as homopolyesters or copolyesters of dicarboxylic acids, alkylene diols, polyalkylene glycols and/or aliphatic hydroxycarboxylic acids; homopolyamides or copolyamides of dicarboxylic acids, alkylenediamines and/or aliphatic aminocarboxylic acids; corresponding polyester-polyamide copolymers, polyanhydrides, polyorthoesters, polyphosphazenes and polycarbonates. The biodegradable polymers may be linear, branched or crosslinked. Specific examples are polyglycolic acid, polylactic acid and poly-d,l-lactic/glycolic acid. Other examples of polymers are water-soluble polymers such as, for example, polyoxaalkylenes (polyoxyethylene, polyoxypropylene and mixed polymers thereof), polyacrylamides and hydroxyl-alkylated polyacrylamides, polymaleic acid and esters or amides thereof, polyacrylic acid and esters or amides thereof, polyvinyl alcohol and esters or ethers thereof, polyvinyl imidazole, polyvinyl pyrrolidone and natural polymers, such as chitosan, for example.

Examples of medicinal carriers include phosphates, such as dicalcium phosphate.

Examples of suitable lubricating agents include natural or synthetic oils, fats, waxes or fatty-acid salts such as magnesium stearate.

Surfactants (surface-active agents) may be anionic, cationic, amphoteric or neutral. Examples of useful surfactants include lecithin, phospholipids, octyl sulfate, decyl sulfate, dodecyl sulfate, tetradecyl sulfate, hexadecyl sulfate and octadecyl sulfate, sodium oleate or sodium caprate, 1-acylaminoethane-2-sulfonic acids such as 1-octanoylaminoethane-2-sulfonic acid, 1-decanoylaminoethane-2-sulfonic acid, 1-dodecanoylaminoethane-2-sulfonic acid, 1-tetradecanoylamino-ethane-2-sulfonic acid, 1-hexadecanoylaminoethane-2-sulfonic acid and 1-octadecanoylaminoethane-2-sulfonic acid, bile acids, salts and derivatives thereof, such as, for example, cholic acid, deoxycholic acid, taurocholic acid, taurodeoxycholic acid and sodium glycocholates, sodium caprate, sodium laurate, sodium oleate, sodium lauryl sulfate, sodium cetyl sulfate, sulfated castor oil, sodium dioctyl sulfosuccinate, cocamidopropyl betaine and lauryl betaine, fatty alcohols, cholesterols, glycerin monostearate or distearate, glycerin monooleate or dioleate, glycerin monopalmitate or dipalmitate and polyoxyethylene stearate.

Examples of suitable sweetening agents include sucrose, fructose, lactose and aspartame.

Examples of useful flavoring agents include peppermint, oil of wintergreen or fruit flavor such as cherry or orange flavor.

Examples of suitable coating agents include gelatins, waxes, shellac, sugars and biodegradable polymers.

Examples of preservation agents include methylparaben or propylparaben, sorbic acid, chlorobutanol and phenol.

Examples of ancillary agents include aromatic principles.

Examples of suitable thickening agents include synthetic polymers, fatty acids, fatty-acid salts, fatty-acid esters and fatty alcohols.

Examples of suitable liquid carriers include water, alcohols (ethanol, glycerol, propylene glycol, liquid polyethylene glycols), polytriazines and oils. Examples of solid carriers are talc, aluminas, microcrystalline cellulose, silicon dioxide, aluminium oxide and similar solid substances.

The composition according to the invention may also contain isotonic agents such as, for example, sugars, physiological buffers and sodium chloride.

The composition according to the invention may also be formulated as an effervescent tablet or effervescent powder which decomposes in an aqueous environment, thereby formulating solutions or suspensions for drinking.

The composition according to the invention may also be a formulation with delayed and/or controlled release of the active substance upon contact with body fluids of the gastrointestinal tract, in order to achieve a substantially constant and effective level of the active substance in the blood plasma.

The crystalline morphine sulfate can also be used together with at least one further pharmaceutical active substance for combination therapies. To this end, at least one further active substance may be additionally dispersed or dissolved in the composition according to the invention.

Yet another aspect of the present invention is a crystalline salt of 7,8-Didehydro-4,5 alpha-epoxy-17-methylmorphinan-3,6 alpha-diol sulfate(2:1) of formula (I), i.e. of morphine sulfate, according to the invention as a medicament. With further reference to the detailed description above, the medicament to the invention may exist as a liquid, semisolid or solid medicinal form, for example in the form of injection solutions, drops, juices, syrups, sprays, suspensions, tablets, patches, capsules, plasters, suppositories, ointments, creams, lotions, gels, emulsions, aerosols, or in multiparticulate form, for example in the form of pellets or granulates, optionally pressed into tablets, filled in capsules or suspended in a liquid, and may also be administered as such.

In a preferred manner, the medicament according to the invention is suitable for the treatment and/or prevention and/or inhibition of pain, preferentially of acute pain, chronic pain, neuropathic pain or visceral pain.

Finally, the present invention is also directed to a crystalline salt of 7,8-Didehydro-4,5 alpha-epoxy-17-methylmorphinan-3,6 alpha-diol sulfate(2:1) according to the invention for the treatment and/or prevention of pain.

In a particularly preferred manner, the crystalline morphine sulfate salt according to the invention is suitable for the treatment and/or inhibition of pain, preferentially of acute pain, chronic pain, neuropathic pain or visceral pain; depressions; epilepsy; Parkinson's disease; abuse of alcohol and/or drugs (in particular, nicotine and/or cocaine) and/or medicaments; dependence on alcohol and/or drugs (in particular, nicotine and/or cocaine) and/or medicaments; preferentially for the prophylaxis and/or reduction of withdrawal symptoms in the case of dependence on alcohol and/or drugs (in particular, nicotine and/or cocaine) and/or medicaments; of the development of tolerance phenomena in relation to medicaments, in particular in relation to opioids, or for anxiolysis.

Lastly, the present invention also encompasses a method of treating pain in a subject in need thereof, said method comprising administering to said subject an analgesically effective amount of a crystalline morphine sulfate according to the invention.

### BRIEF DESCRIPTION OF THE DRAWINGS

The present invention will be further described with reference to the following figures and examples without wishing to be limited in any way.

FIG. 1 shows an XRPD diagram of the first polymorph of morphine sulfate in a formulation;

FIG. 2 shows an XRPD diagram of an embodiment of the second polymorph of morphine sulfate in a formulation;

FIG. 3 shows an XRPD diagram of an embodiment of the second polymorph of morphine sulfate in a formulation;

FIG. 4 shows an XRPD diagram of the third polymorph of morphine sulfate in a formulation;

FIG. 5 shows an XRPD diagram of the fourth polymorph of morphine sulfate in a formulation; and

FIG. 6 shows an XRPD diagram of the fourth polymorph of morphine sulfate in an isolated form.

FIGS. 1 to 6 show XRPD diagrams of polymorphic forms of morphine sulfate. Regarding the diagrams, they were formed by using copper radiation (1.54060 Copper) and a Curved Germanium (111) monochromator.

In detail, the data were generated using a Stoe & Cie X-ray powder diffractometer using Cu Kα radiation, fitted with a fine focus X-ray tube, set at 40 kV and 40 mA and a Germanium monochromator(111). Scan mode was Debye-Scherrer. Diffracted radiation was detected by a linear PSD in 0.5 steps-30 sec/step and 120 sec/step. Continuous scans were used in the 2θ ranges from 5 to 50.

According to FIG. 1 and thus according to the first polymorph, this form may in an exemplarily and non-limiting manner be generated as follows.

The following formulation was used as defined in table 7:

TABLE 7
Formulation used for forming the first polymorph

| per dose [mg] | Substance | Amount [%] |
|---|---|---|
| 95.20 | Morphine sulfate | 36.62 |
| | pentahydrate | |
| 2.11 | Citric acid | 0.81 |
| 24.00 | HPMC K100M Premium | 9.23 |
| 26.17 | PEG 6000 | 10.07 |
| 0.52 | Alpha-Tocopherole | 0.20 |
| 112.00 | PEO 7 Mio. | 43.08 |
| 260.00 | | 100.00 |

The formulation according to table 7 thus comprises the following ingredients: morphine sulfate pentahydrate, which may be commercially available for example by the company Johnson Matthey Macfarlan Smith, Edinburgh, citric acid, Cellulose ether, such as HPMC K 100M premium, available from the Firm Dow Personal Care, polyethyleneglycole, such as with an average molecular weight Mn of 6000 (PEG 6000), alpha tocopherole and polyethyleneoxide, such as with an average molecular weight Mn of 7 million (PEO 7 Mio.).

The formulation according to table 7 was processed as follows.

The extrudates were manufactured in a batch size of about 500 g using the process steps weighing, sieving, blending, extrusion. The weighing was performed on a 1 kg balance. Subsequently the powder was sieved using a sieve with a mesh size of 1.0 mm and blended in a Bohle LM40 MC1 blender (12 rpm, 10 min). The final powder blend was then extruded using a Leistritz ZSE 18 (Twin Screw Extruder).

The extruder comprises an extrusion die (nozzle) diameter of 5 mm and comprises ten different heating zones and a die which could be separately heated. The first heating zine is a feeder and the last heating zine is the nozzle. The speed screw may be adjusted in 80-120 rpm, for example to 100 rpm. The feed rate used may be in the range of 10-20 g/min, for example at 16.66 g/min. The formulation was extruded to result in a strand having a diameter in a range of 5 mm and having a length of 9.4 mm.

The temperature in the extruder was varied from room temperature at the entry to 135° C. at the exit. The first heating zone was adjusted to a temperature within the range of from 20° C. to 30° C., whereas the further heating zones, except the nozzle, which had a temperature of 135° C., were each adjusted to temperatures within the range of from 40° C. to 120° C. in such a manner that an initial temperature increase to 100° C. or above and a subsequent intermediate temperature decrease was realized.

This polymorphic form, or its X-ray powder diffractogram, respectively, according to FIG. 1, comprises one or more of the following reflexes as stated in the following table 8, or it may consist of the following reflexes as stated in the following table 8 (±0,2 in 2θ and recorded using CuK_{α} radiation; the relative intensity I(rel) is 100 at most):

TABLE 8
X-ray powder diffractogram of the first polymorph

| 2θ | I (rel) |
|---|---|
| 8.4 | 24.5 |
| 12.1 | 8.8 |
| 12.5 | 17.4 |
| 13.6 | 56.8 |
| 14.2 | 45.9 |
| 15.2 | 55.2 |
| 16.3 | 17.0 |
| 16.7 | 20.5 |
| 16.9 | 24.4 |
| 17.3 | 9.3 |
| 17.7 | 46.1 |
| 20.0 | 22.8 |
| 20.4 | 19.8 |
| 21.0 | 22.7 |
| 24.2 | 30.1 |
| 24.5 | 21.3 |
| 25.2 | 9.2 |
| 25.5 | 16.9 |
| 25.9 | 19.3 |
| 26.4 | 15.0 |
| 28.4 | 7.6 |
| 29.1 | 10.1 |
| 30.0 | 5.2 |
| 30.7 | 8.9 |
| 31.3 | 4.7 |
| 32.0 | 6.0 |
| 32.9 | 7.8 |
| 33.7 | 7.0 |

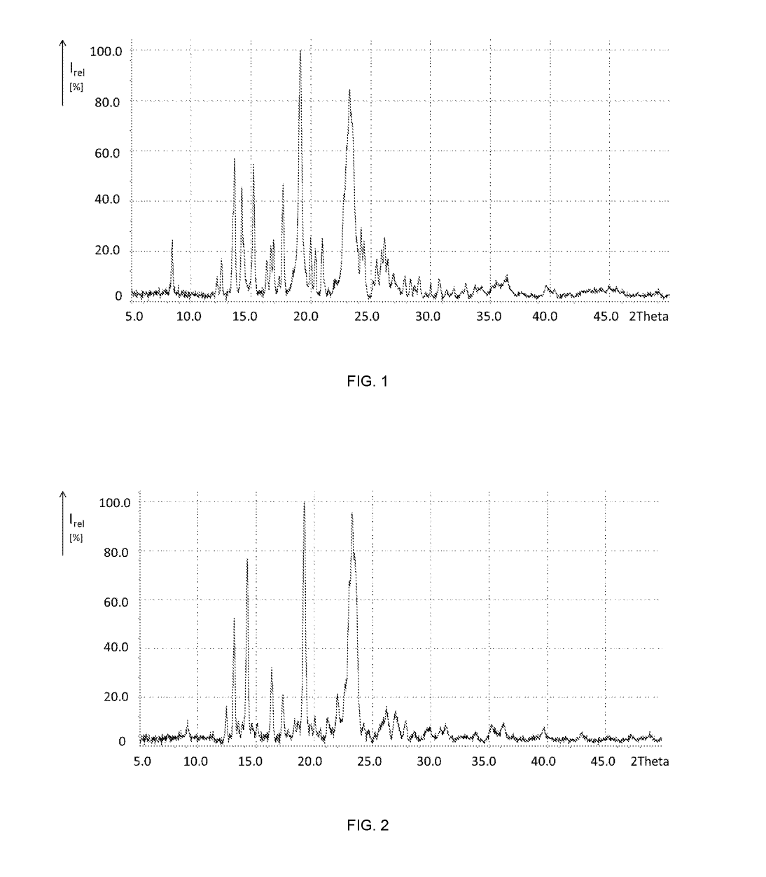

According to FIG. 2 and thus according to the second embodiment of the present invention, the second embodiment relating to the second polymorph, this form may in an exemplarily and non-limiting manner be generated as follows.

The following formulation was used as defined in table 9:

TABLE 9
Formulation used for forming the second polymorph according to the
second embodiment

| per dose [mg] | Substance | Amount [%] |
|---|---|---|
| 200 | Morphine sulfate | 40 |
| | pentahydrate | |
| 4 | Citric acid | 0.8 |
| 36 | HPMC K100M Premium | 7.2 |
| 50 | PEG 6000 | 10 |
| 1 | α-Tocopherole | 0.2 |
| 159 | PEO 7 Mio. | 31.8 |
| 50 | Carbopol | 10 |
| 500 | | 100.00 |

The formulation according to table 9 thus comprises the following ingredients: morphine sulfate pentahydrate, which may be commercially available for example by the company Johnson Matthey Macfarlan Smith, Edinburgh, citric acid, Cellulose ether, such as HPMC K 100M premium, available from the Firm Dow Personal Care, polyethyleneglycole, such as with an average molecular weight Mn of 6000 (PEG 6000), alpha tocopherole, polyethyleneoxide, such as with an average molecular weight Mn of 7 million (PEO 7 Mio.), and Carbopol, such as Carbopol 71G NF, commercially available by the firm Lubrizol.

The formulation according to table 9 was processed as follows.

The extrudates were manufactured in a batch size of about 500 g using the process steps weighing, sieving, blending, extrusion. The weighing was performed on a 1 kg balance. Subsequently the powder was sieved using a sieve with a mesh size of 1.0 mm and blended in a Bohle LM40 MC1 blender (12 rpm, 10 min). The final powder blend was then extruded using a Leistritz ZSE 18 (Twin Screw Extruder).

The extruder comprises an extrusion die (nozzle) diameter of 6 mm and comprises eleven heating zones, wherein the 11^{th} heating zone is the nozzle and wherein the first heating zone is the feeding portion. The speed of the screw or of the two screws may be adjusted to 80-120 rpm, for example to 100 rpm. The feed rate used may be in the range of 10-20 g/min, for example at 16.66 g/min. The formulation was extruded to result in a strand having a diameter in a range of 6 mm and having a length of 15 mm.

The temperature in the extruder was varied from room temperature at the entry to 140° C. at the exit. The first heating zone was adjusted to a temperature within the range of from 20° C. to 30° C., whereas the further heating zones, except the nozzle, which had a temperature of 140° C., were each adjusted to temperatures within the range of from 80° C. to 120° C. in such a manner that an initial temperature increase to 100° C. or above and a subsequent intermediate slight temperature decrease was realized.

This polymorphic form or its X-ray powder diffractogram according to FIG. 2, comprises one or more of the following reflexes as stated in the following table 10, or it may consist of the following reflexes as stated in the following table 10 (±0,2 in 2θ and recorded using CuK_{α} radiation; the relative intensity I(rel) is 100 at most):

TABLE 10
X-ray powder diffractogram of the second polymorph according to the
second embodiment

| 2θ | I (rel) |
|---|---|
| 9.1 | 10.9 |
| 12.4 | 16.4 |
| 13.1 | 52.0 |
| 14.2 | 74.1 |
| 16.3 | 32.0 |
| 17.3 | 21.0 |
| 20.1 | 12.1 |
| 24.2 | 8.5 |
| 24.7 | 4.9 |

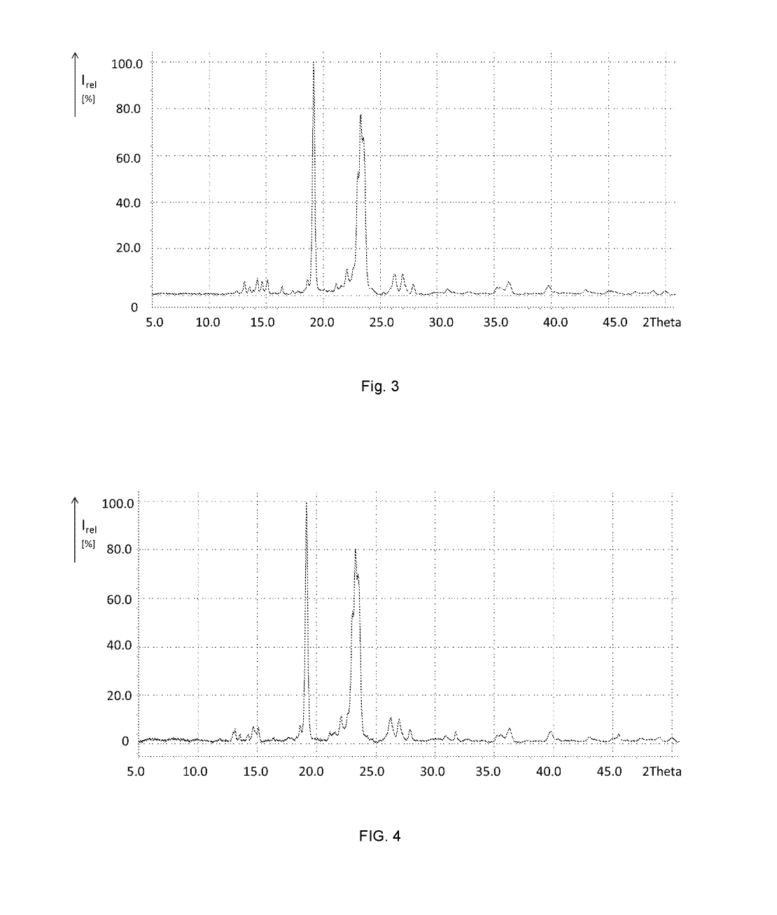

According to FIG. 3 and thus according to the second polymorph according to the third embodiment of the present invention, this form may in an exemplarily and non-limiting manner be generated as follows.

The following formulation was used as defined in table 11:

TABLE 11
Formulation used for forming the second polymorph according to the third
embodiment

| per dose [mg] | Substance | Amount [%] |
|---|---|---|
| 10 | Morphine sulfate | 5.56 |
| | pentahydrate | |
| 1.44 | Citric acid | 0.80 |
| 25.20 | PEG 6000 | 14.00 |
| 0.36 | α-Tocopherole | 0.20 |
| 143.00 | PEO 7 Mio. | 79.44 |
| 180.00 | | 100.00 |

The formulation according to table 11 thus comprises the following ingredients: morphine sulfate pentahydrate, which may be commercially available for example by the company Johnson Matthey Macfarlan Smith, Edinburgh, citric acid, polyethyleneglycole, such as with an average molecular weight Mn of 6000 (PEG 6000), alpha tocopherole and polyethyleneoxide, such as with an average molecular weight Mn of 7 million (PEO 7 Mio.).

The formulation according to table 11 was processed as follows.

The extrudates were manufactured in a batch size of about 500 g using the process steps weighing, sieving, blending, extrusion. The weighing was performed on a 1 kg balance. Subsequently the powder was sieved using a sieve with a mesh size of 1.0 mm and blended in a Bohle LM40 MC1 blender (12 rpm, 10 min). The final powder blend was then extruded using a Leistritz ZSE 18 (Twin Screw Extruder).

The extruder comprises an extrusion die (nozzle) diameter of 0.8 mm and comprises ten heating zones, wherein the 10^{th} heating zone is the nozzle and wherein the first heating zone is the feeding portion. The speed of the screw or of the two screws may be adjusted to 80-120 rpm, for example to 100 rpm. The feed rate used may be in the range of 10-20 g/min, for example at 16.66 g/min. The formulation was extruded to result in pellets having a diameter in a range of 0.8-1.2 mm, particularly of 0.8 mm

The temperature in the extruder was steadily increased from room temperature at the entry to 133° C. at the exit, whereas the first heating zone was adjusted to a temperature within the range of from 20° C. to 30° C. and the following heating zones were each adjusted to temperatures within the range of from 80° C. to 133° C. in a manner such that a steady temperature increase was realized.

This polymorphic form or its X-ray powder diffractogram according to FIG. 3, comprises one or more of the following reflexes as stated in the following table 12, or it may consist of the following reflexes as stated in the following table 12 (±0,2 in 2θ and recorded using CuK_{α} radiation; the relative intensity I(rel) is 100 at most):

TABLE 12
X-ray powder diffractogram of the second polymorph according to the
third embodiment

| 2θ | I (rel) |
|---|---|
| 9.1 | 1 |
| 12.4 | 1.5 |
| 14.2 | 7.3 |
| 16.4 | 3.9 |
| 17.3 | 2.0 |
| 17.8 | 1.9 |
| 20.1 | 2.8 |
| 20.6 | 2.3 |

According to FIG. 4 and thus according to the third polymorph, this form may in an exemplarily and non-limiting manner be generated as follows.

The following formulation was used as defined in table 13:

TABLE 13
Formulation used for forming the third polymorph

| per dose [mg] | Substance | Amount [%] |
|---|---|---|
| 10.00 | Morphine sulfate | 5.56 |
| | pentahydrate | |
| 1.44 | Citric acid | 0.80 |
| 25.20 | PEG 6000 | 14.00 |
| 0.36 | α-Tocopherole | 0.20 |
| 98.00 | PEO 7 Mio. | 54.44 |
| 9.00 | Xanthan | 5.00 |
| 36.00 | Carboxy methyl starch | 20.00 |
| 180.00 | | 100.00 |

The formulation according to table 13 thus comprises the following ingredients: morphine sulfate pentahydrate, which may be commercially available for example by the company Johnson Matthey Macfarlan Smith, Edinburgh, citric acid, polyethyleneglycole, such as with an average molecular weight Mn of 6000 (PEG 6000), alpha tocopherole, polyethyleneoxide, such as with an average molecular weight Mn of 7 million (PEO 7 Mio.), xanthane as well as carboxy methyl starch.

The formulation according to table 13 was processed as follows.

The extrudates were manufactured in a batch size of about 500 g using the process steps weighing, sieving, blending, extrusion. The weighing was performed on a 1 kg balance. Subsequently the powder was sieved using a sieve with a mesh size of 1.0 mm and blended in a Bohle LM40 MC1 blender (12 rpm, 10 min). The final powder blend was then extruded using a Leistritz ZSE 18 (Twin Screw Extruder).

The extruder comprises an extrusion die (nozzle) with a diameter of 0.8 mm and comprises ten heating zones, wherein the 10^{th} heating zone is the nozzle and wherein the first heating zone is the feeding portion. The speed of the screw or of the two screws may be adjusted to 80-120 rpm, for example to 100 rpm. The feed rate used may be in the range of 10-20 g/min, for example at 16.66 g/min. The formulation was extruded to result in pellets having a diameter in a range of 0.8-1.2 mm, particularly of 0.8 mm.

The temperature in the extruder was steadily increased from room temperature at the entry to 130° C. at the exit, whereas the first heating zone was adjusted to a temperature within the range of from 20° C. to 30° C. and the following heating zones were each adjusted to temperatures within the range of from 80° C. to 130° C. in a manner such that a steady temperature increase was realized.

This polymorphic form, or its X-ray powder diffractogram, respectively, according to FIG. 4, comprises one or more of the following reflexes as stated in the following table 14, or it may consist of the following reflexes as stated in the following table 14 (±0,2 in 2θ and recorded using CuK_{α} radiation; the relative intensity I(rel) is 100 at most):

TABLE 14
X-ray powder diffractogram of the third polymorph

| 2θ | I (rel) |
|---|---|
| 11.9 | 2.4 |
| 13.0 | 5.0 |
| 14.3 | 3.9 |
| 14.9 | 5.4 |
| 16.1 | 1.4 |
| 16.4 | 2.1 |
| 18.3 | 3.2 |
| 31.7 | 5.1 |

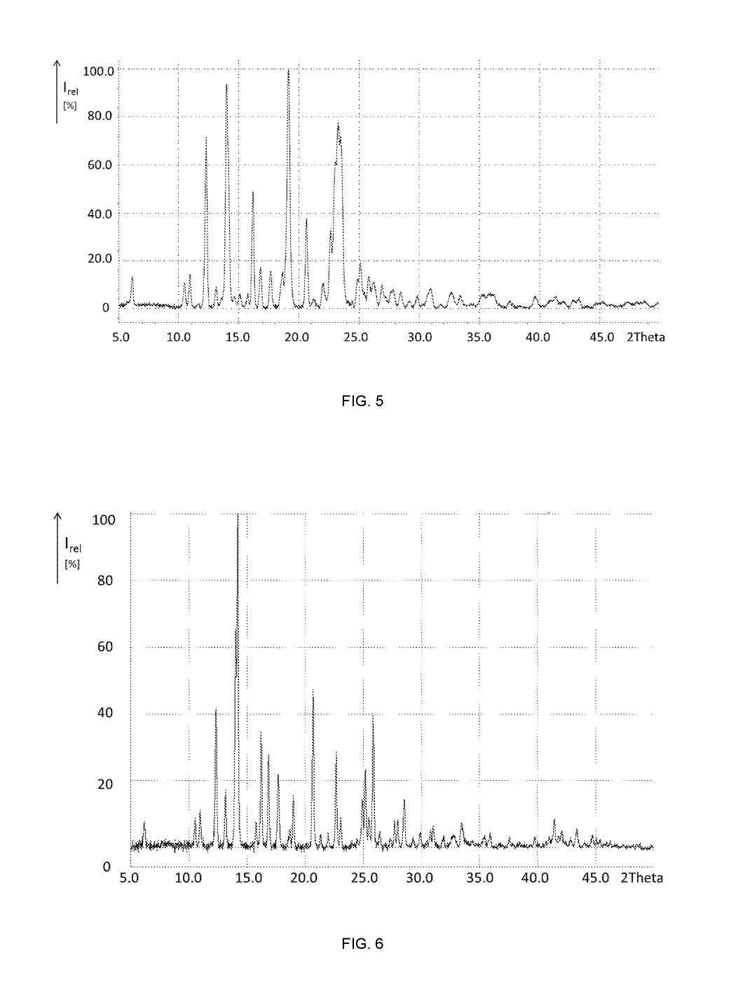

According to FIG. 5 and thus according to the fourth polymorphic form of morphine sulfate according to the fifth embodiment, this form may in an exemplarily and non-limiting manner be generated as follows.

Firstly, the extrudate according to the above-defined first embodiment is generated. Such extrudate was processed in a DVS experiment (Dynamic-Vapour-Sorption). The extrudate was processed with a distinct humidity program. The probe was processed at a comparably higher relative humidity, e.g. 80% rH to 95% rH, for example at 90% rH for a time range of 10 to 15 hours, e.g. of 12 hours. Afterwards, the humidity was decreased and the probe was processed at a comparably lower relative humidity, e.g. 5% rH to 15% rH, for example at 10% rH for a time range of 10 to 15 hours, e.g. of 12 hours. The formed substance was morphine sulfate according to the fifth embodiment. As such, the form provided was present in a formulation. The device used was a DVS-1000, commercially available by the firm Surface Measurement Systems.

This polymorphic form or its X-ray powder diffractogram according to FIG. 5, comprises one or more of the following reflexes as stated in the following table 15, or it may consist of the following reflexes as stated in the following table 15 (±0,2 in 2θ and recorded using CuK_{α} radiation; the relative intensity I(rel) is 100 at most):

TABLE 15
X-ray powder diffractogram of the fourth polymorph according to the fifth
embodiment

| 2θ | I (rel) |
|---|---|
| 6.1 | 13.2 |
| 10.5 | 10.9 |
| 10.9 | 14.4 |
| 11.7 | 2.0 |
| 12.3 | 68.6 |
| 13.1 | 8.8 |
| 14.0 | 94.7 |
| 15.7 | 5.6 |
| 16.2 | 48.6 |
| 16.8 | 17.1 |
| 17.6 | 15.6 |
| 20.6 | 37.1 |
| 21.2 | 4.2 |
| 22.0 | 9.8 |
| 25.1 | 18.3 |
| 25.8 | 13.4 |
| 28.5 | 7.2 |
| 29.1 | 2.8 |
| 29.8 | 4.5 |

According to FIG. 6 and thus according to the isolated fourth polymorphic form of morphine sulfate according the sixth embodiment, this form may in an exemplarily and non-limiting manner be generated as follows.

Morphine sulfate pentahydrate was processed in a DVS experiment (Dynamic-Vapour-Sorption. The morphine sulfate pentahydrate used is commercially available for example by the company Johnson Matthey Macfarlan Smith, Edinburgh. It was processed with a distinct humidity program. The probe was processed at a comparably higher relative humidity, e.g. 80% rH to 95% rH, for example at 90% rH for a time range of 10 to 15 hours, e.g. at 12 hours. Afterwards, the humidity was decreased and the probe was processed at a comparably lower relative humidity, e.g. 5% rH to 15% rH, for example at 10% rH for a time range of 10 to 15 hours, e.g. at 12 hours. The device used was a DVS-1000, commercially available by the firm Surface Measurement Systems.

This polymorphic form or its X-ray powder diffractogram according to FIG. 6, comprises one or more of the following reflexes as stated in the following table 16, or it may consist of the following reflexes as stated in the following table 16 (±0,2 in 2θ and recorded using CuK_{α} radiation; the relative intensity I(rel) is 100 at most):

TABLE 16
X-ray powder diffractogram of the fourth polymorph according to the sixth
embodiment

| 2θ | I (rel) |
|---|---|
| 6.1 | 6.9 |
| 10.5 | 8.9 |
| 11.0 | 10.1 |
| 12.3 | 43.1 |
| 13.1 | 17.5 |
| 14.0 | 67.3 |
| 14.2 | 100 |
| 15.8 | 7.5 |
| 16.2 | 35.4 |
| 16.8 | 26.8 |
| 17.7 | 21.7 |
| 18.7 | 5.3 |
| 19.0 | 15.4 |
| 20.7 | 48.5 |
| 21.3 | 3.2 |
| 22.0 | 3.6 |
| 22.7 | 28.5 |
| 23.1 | 8.5 |
| 24.4 | 1.9 |
| 24.9 | 16.4 |
| 25.1 | 23.4 |
| 25.5 | 8.7 |
| 25.8 | 38.4 |
| 26.4 | 5.0 |
| 27.4 | 2.2 |
| 27.7 | 8.1 |
| 28.0 | 7.8 |
| 28.5 | 14.6 |
| 29.2 | 1.4 |
| 29.9 | 3.3 |
| 30.5 | 1.5 |
| 30.8 | 4.6 |
| 31.0 | 5.7 |
| 31.9 | 2.3 |
| 32.8 | 3.5 |
| 33.4 | 6.9 |
| 35.4 | 2.9 |
| 35.9 | 4.4 |
| 37.6 | 3.0 |
| 39.8 | 2.7 |
| 41.5 | 8.6 |

1. A crystalline, salt of 7,8-Didehydro-4,5 alpha-epoxy-17-methylmorphinan-3,6 alpha-diol sulfate(2:1) according to formula (I): 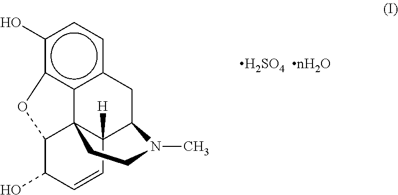
wherein the X-ray powder diffractogram of the salt comprises one or more of the following reflexes (±0,2 in 2θ): 13.6, 15.2, 17.7, or wherein the X-ray powder diffractogram of the salt comprises one or more of the following reflexes (±0,2 in 2θ): 13.1, 14.2, 16.3, or wherein the X-ray powder diffractogram of the salt comprises one or more of the following reflexes (±0,2 in 2θ): 14.2, 16.4, 20.1, or wherein the X-ray powder diffractogram of the salt comprises one or more of the following reflexes (±0,2 in 2θ): 13.0, 14.9, 31.7, or wherein the X-ray powder diffractogram of the salt comprises one or more of the following reflexes (±0,2 in 2θ): 12.3, 14.0, 16.2, or wherein the X-ray powder diffractogram of the salt comprises one or more of the following reflexes (±0,2 in 2θ): 14.0, 14.2, 20.7.

2. The salt according to claim 1, wherein the X-ray powder diffractogram of the salt comprises one or more of the following reflexes (±0,2 in 2θ): 13.6, 14.2, 15.2, 17.7, 24.2.

3. The salt according to claim 2, wherein the X-ray powder diffractogram of the salt comprises one or more of the following reflexes (±0,2 in 2θ): 8.4, 12.1, 12.5, 13.6, 14.2, 15.2, 16.3, 16.7, 16.9, 17.3, 17.7, 20.0, 20.4, 21.0, 24.2, 24.5, 25.2, 25.5, 25.9, 26.4, 28.4, 29.1, 30.0, 30.7, 31.3, 32.0, 32.9, 33.7.

4. The salt according to claim 1, wherein the X-ray powder diffractogram of the salt comprises one or more of the following reflexes (±0,2 in 2θ): 12.4, 13.1, 14.2, 16.3, 17.3, 20.1.

5. The salt according to claim 4, wherein the X-ray powder diffractogram of the salt comprises one or more of the following reflexes (±0,2 in 2θ): 9.1, 12.4, 13.1, 14.2, 16.3, 17.3, 20.1, 24.2, 24.7.

6. The salt according to claim 1, wherein the X-ray powder diffractogram of the salt comprises one or more of the following reflexes (±0,2 in 2θ): 14.2, 16.4, 17.3, 20.1, 20.6.

7. The salt according to claim 6, wherein the X-ray powder diffractogram of the salt comprises one or more of the following reflexes (±0,2 in 2θ): 9.1, 12.4, 14.2, 16.4, 17.3, 17.8, 20.1, 20.6.

8. The salt according to claim 1, wherein the X-ray powder diffractogram of the salt comprises one or more of the following reflexes (±0,2 in 2θ): 13.0, 14.3, 14.9, 18.3, 31.7.

9. The salt according to claim 8, wherein the X-ray powder diffractogram of the salt comprises one or more of the following reflexes (±0,2 in 2θ): 11.9, 13.0, 14.3, 14.9, 16.1, 16.4, 18.3, 31.7.

10. The salt according to claim 1, wherein the X-ray powder diffractogram of the salt comprises one or more of the following reflexes (±0,2 in 2θ): 12.3, 14.0, 16.2, 16.8, 20.6, 25.1.

11. The salt according to claim 10, wherein the X-ray powder diffractogram of the salt comprises one or more of the following reflexes (±0,2 in 2θ): 6.1, 10.5, 10.9, 11.7, 12.3, 13.1, 14.0, 15.7, 16.2, 16.8, 17.6, 20.6, 21.2, 22.0, 25.1, 25.8, 28.5, 29.1, 29.8.

12. The salt according to claim 1, wherein the X-ray powder diffractogram of the salt comprises one or more of the following reflexes (±0,2 in 2θ): 12.3, 14.0, 14.2, 16.2, 20.7, 25.8.

13. The salt according to claim 12, wherein the X-ray powder diffractogram of the salt comprises one or more of the following reflexes (±0,2 in 2θ): 6.1, 10.5, 11.0, 12.3, 13.1, 14.0, 14.2, 15.8, 16.2, 16.8, 17.7, 18.7, 19.0, 20.7, 21.3, 22.0, 22.7, 23.1, 24.5, 24.9, 25.2, 25.5, 25.8, 26.4, 27.4, 27.7, 28.0, 28.5, 29.2, 29.9, 30.5, 30.8, 31.0, 31.9, 32.8, 33.4, 35.4, 35.9, 37.6, 39.8, 41.5.

14. A pharmaceutical composition comprising an amount of a crystalline salt of 7,8-Didehydro-4,5 alpha-epoxy-17-methylmorphinan-3,6 alpha-diol sulfate(2:1) of formula (I) according to claim 1.

15. A method of treating and/or preventing pain in a patient in need thereof, said method comprising administering to said patient an effective amount therefor of a crystalline salt of 7,8-Didehydro-4,5 alpha-epoxy-17-methylmorphinan-3,6 alpha-diol sulfate(2:1) of formula (I) according to claim 1.
